(19) United States
(12) Patent Application Publication
(10) Pub. No.: US 2026/0193162 A1
(43) Pub. Date: Jul. 9, 2026

(54) CORROSION METAL REMOVAL FROM ETHYL ACETATE PRODUCTION PROCESS

(71) Applicant: SAHARA INTERNATIONAL PETROCHEMICAL COMPANY (SIPCHEM), Dhahran (SA)

(72) Inventors: Sekhar B. MAMILLA, Dhahran (SA); Lesibana P. LEDWABA, Dhahran (SA); Abdullah S. ABUJOHNAH, Dhahran (SA); Tahir Iqbal ALVI, Dhahran (SA); Mohammed ASHFAQUDDIN, Dhahran (SA)

(73) Assignee: SAHARA INTERNATIONAL PETROCHEMICAL COMPANY (SIPCHEM), Dhahran (SA)

(21) Appl. No.: 19/014,914

(22) Filed: Jan. 9, 2025

(51) Int. Cl. C07C 67/54

(52) U.S. Cl. CPC C07C 67/54

(57) ABSTRACT

A method of producing ethyl acetate, the method including: reacting ethanol, acetic acid, and an acid catalyst to form a product stream; distilling the product stream to form a top stream and a bottom stream including at least one corrosion metal in the bottom stream; cooling the bottom stream to precipitate at least a first portion of the corrosion metal and form a supernatant stream; contacting the supernatant stream with a cation exchange resin to remove a second portion of the corrosion metal and form a reduced metal stream; evaporating the reduced metal stream to purge at least a third portion of the corrosion metal from the reduced metal stream and form a purged stream; and returning the purged stream to the distillation column, where the bottom stream is cooled to a temperature of about 45° C. or less in the settling tank.

### BACKGROUND

### Technical Field

The present disclosure relates to methods of removing corrosion metals from process streams in ethyl acetate production. The present disclosure also relates to methods of removing sulfate ions from process streams in ethyl acetate production. The present disclosure also relates to methods of removing the corrosion metals and the sulfate ions using precipitation and/or cation exchange resins at low temperatures.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Ethyl acetate is an important raw material in the chemicals industry, with an annual global market of more than $4 billion, which is growing at a faster rate than global gross domestic product (GDP). It is a flammable, colorless liquid used in glues, nail polish removers, and other consumer products, as well as being an important process solvent in the pharmaceutical industry, and used for the decaffeination of tea and coffee. It is also an important industrial solvent, with uses in paints, varnishes, lacquers, cleaning mixtures, colorants, and artificial fibers.

Currently, as a major industrial route ethyl acetate is produced via a Fischer esterification reaction from ethanol and acetic acid, in the presence of strongly acidic homogeneous catalysts. The use of high temperatures, typically greater than 110° C., in combination with the presence of both strong acids and water (by-product), lead to major problems with corrosion of the stainless steel equipment used in ethyl acetate production plants. This leads to the need for highly corrosion-resistant construction materials and frequent maintenance and replacement of equipment, which leads to high capital expenditures. In addition, the corrosion metals cause side reactions that leads to the formation of undesirable chemical by-products which reduces the yield and product quality issues.

In addition to the corrosion metals, another challenge occurring in ethyl acetate production is that, during certain high-temperature distillation and vaporization processes, the sulfur-containing acid catalyst can decompose into sulfate ions. The sulfate ions can further convert into sulfuric acid vapors by reacting with water vapor also present in the process streams. The combination of sulfuric acid, acetic acid and water, along with high process temperatures of 115° C. or higher, can further corrode metal plant equipment, leading to frequent maintenance and replacement costs.

To protect the process equipment from corrosion during the starting up of the plant, a special passivation process is followed to develop a protective layer. When the concentration of the corrosion metal crosses a critical limit, for example from 0.1 wt % to 2 wt %, the high concentration of corrosion metals start damaging the protection layer. In some embodiments, the critical limit may be about 0.001 wt %, about 0.005 wt %, about 0.01 wt %, about 0.05 wt %, about 0.1 wt %, about 0.25 wt %, about 0.5 wt %, about 0.75 wt %, about 1.0 wt %, about 1.25 wt %, about 1.5 wt %, about 1.75 wt %, or about 2 wt %. When the protection layer is damaged, the corrosion rate can increase exponentially, thus adding more corrosion metals into the circulating process liquid. This eventually leads to piping and process equipment damage, creating unsafe process conditions including process leaks.

Because of the above reasons, there is a need for improved technologies to maintain the corrosion metals concentrations within the critical limit, such as below from about 0.001 wt % to about 0.5 wt %, and to thereby reduce the need to replace the equipment regularly. Accordingly, one object of the present disclosure is to provide methods of producing ethyl acetate with controlled levels of corrosion metals.

### SUMMARY

Some embodiments of the present disclosure relate to a method of producing ethyl acetate. In some embodiments, the method includes: reacting ethanol, acetic acid, and an acid catalyst in an esterification reactor to form a product stream including the ethyl acetate, water, and at least one corrosion metal; distilling the product stream in a distillation column to form a top stream including the ethyl acetate, ethanol and a water azeotrope, and a bottom stream including acetic acid, ethanol, ethyl acetate, acid catalyst, water, and the at least one corrosion metal. Due to the combination of acidic environment, water and high temperature cause the corrosion metals leaching out of the process equipment into the process liquid. In some embodiments, the method includes cooling the bottom stream in a settling tank to precipitate at least a first portion of the at least one corrosion metal from the bottom stream and form a supernatant stream; contacting the supernatant stream with a cation exchange resin to remove at least a second portion of the at least one corrosion metal from the supernatant stream and form a reduced metal stream; evaporating a portion of the reduced metal stream in an evaporator tank to purge at least a third portion of the at least one corrosion metal from the reduced metal stream and form a purged stream; and returning at least a portion of the purged stream to the distillation column, where: the bottom stream is cooled to a temperature of about 45° C. or less in the settling tank, and the cooling of the bottom stream and the contacting of the supernatant stream with the cation exchange resin removes at least 75% of the at least one corrosion metal relative to the initial corrosion metal concentration in the bottom stream.

In some embodiments, the method includes reacting ethanol, acetic acid, and an acid catalyst in an esterification reactor to form a product stream including the ethyl acetate and water; distilling the product stream in a distillation column to form a top stream including the ethyl acetate, ethanol and water azeotrope and a bottom stream including acetic acid, ethyl acetate, ethanol, water and acid catalyst. The bottom stream can also include corrosion metals that are leached out of the process equipment and piping due to high temperatures, and process conditions including acetic acid and water. The increase of corrosion metals to the critical levels will damage the corrosion protection layer that is formed during the startup of the process.

To maintain the corrosion metals below the critical levels, there is a need to purge them from the system, such as by cooling at least a part of the bottom stream in a settling tank to precipitate at least a first portion of the at least one corrosion metal from the bottom stream and form a supernatant stream; contacting the supernatant stream with a cation exchange resin to remove at least a second portion of the at least one corrosion metal from the supernatant stream and form a reduced metal stream; evaporating a portion of the reduced metal stream in an evaporator tank to purge at least a third portion of the at least one corrosion metal from the reduced metal stream and form a purged stream; and returning at least a portion of the purged stream to the distillation column, where: the bottom stream is cooled to a temperature of about 45° C. or less in the settling tank, and the cooling of the bottom stream and the contacting of the supernatant stream with the cation exchange resin removes at least 75% of the at least one corrosion metal relative to the initial corrosion metal concentration in the bottom stream.

A method of producing ethyl acetate, the method including reacting ethanol, acetic acid, and an acid catalyst in an esterification reactor to form a product stream including the ethyl acetate, water, acid catalyst and unconverted acetic acid and ethanol; distilling the product stream in a distillation column to form a top stream including the ethyl acetate, ethanol and water azeotrope and a bottom stream including acetic acid, ethyl acetate, water, ethanol and acid catalyst. The corrosion metals, leached out of the process equipment due to corrosive nature of the process liquid and high temperature, are accumulated in the bottom stream of the distillation column. To maintain the corrosion metal concentration below a critical limit, part of the bottom stream is cooled and sent to a settling tank to precipitate out at least part of corrosion metals in salt form. The supernatant liquid is passed through a cation exchange resin to remove a second portion of the corrosion metals before sending it to the evaporation vessels. In the evaporation vessels, a third portion of the corrosion metals are removed by evaporating the liquid and purging the heavy purge with the third portion of corrosion metals.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 illustrates a process flow diagram of a conventional method of producing ethyl acetate.

FIG. 2 illustrates a process flow diagram of a method of producing ethyl acetate, according to some embodiments.

### DETAILED DESCRIPTION

In the drawings, like reference numerals designate identical or corresponding parts throughout the several views. Further, as used herein, the words “a”, “an” and the like generally carry a meaning of “one or more”, unless stated otherwise.

Furthermore, the terms “approximately,” “approximate,” “about,” and similar terms generally refer to ranges that include the identified value within a margin of 20%, 10%, or preferably 5%, and any values therebetween.

As used herein, the term “azeotrope,” “azeotropic mixture,” and the like, means a mixture of two or more liquids or gases that has a constant composition and boiling point during distillation. “Azeotropic distillation” means distillation of an azeotropic mixture.

As used herein, the term “corrosion metal” means a metal ion or salt, or metal-containing compound, which is generally obtained from a metal component, such as a stainless steel tank in an ethyl acetate production plant, through corrosion, oxidation, or other degradation of the metal component. The corrosion metals may include iron, chromium, nickel, aluminum, zinc, titanium, chromium, niobium, copper, and/or molybdenum. In some embodiments, the corrosion metals are oxides or hydroxide salts of the corrosion metal. Typical corrosion metals concentrations in some embodiments are from about 0.001 wt % to about 0.3 wt %, such as about 0.005 wt %, about 0.01 wt %, about 0.05 wt %, about 0.1 wt %, about 0.2 wt %, or about 0.3 wt %.

As used herein, the term “cation exchange resin” means an insoluble material that selectively attracts and exchanges positively charged ions from a solution. Generally, a cation exchange resin comprises a polymeric material that is chemically conjugated to negatively charged functional groups, which bind and trap cationic ions from the solution while allowing neutral and anionic species in the solution to pass through.

Currently, ethyl acetate is produced via a Fischer esterification reaction from ethanol and acetic acid, in the presence of liquid inorganic acid and/or organic acid homogeneous catalysts. The process includes reaction in an esterification reactor, azeotropic distillation, and final purification comprising multiple decanter and/or distillation steps. The reasons for this complex flow scheme include that esterification is an equilibrium-limited reaction, requiring excesses of starting reactants to obtain high yields and thus multicomponent product streams. Additionally, the ethyl acetate, water, and ethanol present during esterification forms binary and ternary azeotropes.

The raw materials, acetic acid and ethanol, used in connection with the process of this invention may be derived from any suitable source including natural gas, petroleum, coal, biomass, and so forth. As examples, acetic acid may be produced via methanol carbonylation, acetaldehyde oxidation, ethylene oxidation, oxidative fermentation, and anaerobic fermentation. Methanol carbonylation processes suitable for production of acetic acid are described in U.S. Pat. Nos. 7,208,624; 7,115,772; 7,005,541; 6,657,078; 6,627,770; 6,143,930; 5,599,976; 5,144,068; 5,026,908; 5,001,259; and 4,994,608, the entire disclosures of which are incorporated herein by reference.

As petroleum and natural gas prices fluctuate becoming either more or less expensive, methods for producing acetic acid and intermediates such as ethanol from alternate carbon sources have drawn increasing interest. In particular, when petroleum is relatively expensive, it may become advantageous to produce acetic acid from synthesis gas (“syngas”) that is derived from more available carbon sources. U.S. Pat. No. 6,232,352, the entirety of which is incorporated herein by reference, for example, teaches a method of retrofitting a methanol plant for the manufacture of acetic acid. By retrofitting a methanol plant, the large capital costs associated with CO generation for a new acetic acid plant are significantly reduced or largely eliminated. All or part of the syngas is diverted from the methanol synthesis loop and supplied to a separator unit to recover CO, which is then used to produce acetic acid.

In some embodiments, some or all of the raw materials for the above-described ethyl acetate production process may be derived partially or entirely from syngas. For example, the acetic acid may be formed from methanol and carbon monoxide, both of which may be derived from syngas. In a similar manner, ethanol may be supplied from syngas. The syngas may be formed by partial oxidation reforming or steam reforming, and the carbon monoxide may be separated from syngas. The syngas, in turn, may be derived from variety of carbon sources. The carbon source, for example, may be selected from the group consisting of natural gas, oil, petroleum, coal, biomass, and combinations thereof. Syngas may also be obtained from bio-derived methane gas, such as bio-derived methane gas produced by landfills or agricultural waste.

In another embodiment, the acetic acid used in the hydrogenation step may be formed from the fermentation of biomass. The fermentation process preferably utilizes an acetogenic process or a homoacetogenic microorganism to ferment sugars to acetic acid producing little, if any, carbon dioxide as a by-product. The carbon efficiency for the fermentation process preferably is greater than 70%, greater than 80% or greater than 90% as compared to conventional yeast processing, which typically has a carbon efficiency of about 67%. Exemplary fermentation processes for forming acetic acid are disclosed in U.S. Pat. Nos. 6,509,180; 6,927,048; 7,074,603; 7,507,562; 7,351,559; 7,601,865; 7,682,812; and 7,888,082, the entireties of which are incorporated herein by reference.

As one way of overcoming the equilibrium limitation, conventional technologies operate with excess amount of acetic acid. This excess amount of acetic acid, along with water, acid catalyst, and high temperatures in some processing streams, such as above 115° C., significantly increases the corrosion rate in the plant even with stainless steel equipment. Because of the equilibrium limitation, ethyl acetate production systems continuously remove products to shift equilibrium forward to increase conversion. The water byproduct is also removed via distillation to enhance the overall conversion by shifting equilibrium.

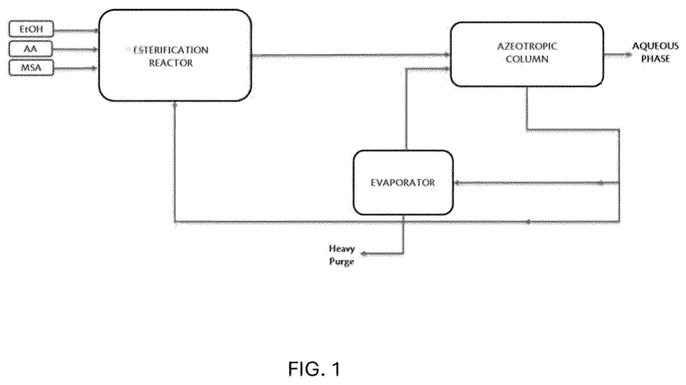

A process flow diagram for a conventional ethyl acetate production process is illustrated in FIG. 1. Ethanol (EtOH), acetic acid (AA), and an acid catalyst such as methanesulfonic acid (MSA) are metered into an esterification reactor. In the esterification reactor, in the presence of the catalyst, ethyl acetate and water are generated by steam heating the mixture of ethanol and acetic acid, at temperatures above 100° C., and often above 125° C., to improve the kinetics.

In some embodiments, the feed ratios of acetic acid, ethanol, and acid catalyst entering the esterification reactor are as shown in Table 1.

TABLE 1
Feed composition for ethyl acetate production

| | Concentration | Concentration | Concentration |
|---|---|---|---|
| | (weight %) | (weight %) | (weight %) |
| Acetic acid | 10 to 90 | 20 to 80 | 30 to 70 |
| Ethanol | 5 to 50 | 10 to 40 | 15 to 30 |
| Methanesulfonic | 0.001 to 5 | 0.01 to 4 | 0.02 to 2 |
| acid (MSA) | | | |

In some embodiments, the amount of ethanol in the product stream exiting the esterification reactor is less than 20 wt % of the mass of the product stream, such as less than 15 wt %, less than 10 wt %, or less than 5 wt %, or less than 1 wt %.

In some embodiments, the amount of acetic acid in the product stream exiting the esterification reactor is less than 20 wt % of the mass of the product stream, such as less than 15 wt %, less than 10 wt %, or less than 5 wt %, or less than 1 wt %.

In some embodiments, the amount of water in the product stream exiting the esterification reactor is less than 20 wt % of the mass of the product stream, such as less than 15 wt %, less than 10 wt %, or less than 5 wt %, or less than 1 wt %. In some embodiments, a portion of the water is removed from the esterification reactor as steam in order to shift the equilibrium forward.

The product from the reactor enters a distillation column as illustrated in FIG. 1. In the distillation column, the ethyl acetate, ethanol and a water azeotrope are evaporated from the top of the column and subsequently condensed to a liquid phase that needs further purification to obtain pure ethanyl acetate product, as shown in FIG. 1.

As shown in FIG. 1, a bottom stream of the azeotropic distillation column can be removed from the distillation column and recycled back to the esterification reactor. The bottom stream generally includes the distillation residue, which may include leftover reactants, by-products of the esterification, and other impurities including the corrosion metals.

The top and bottom streams from the distillation column can be processed in a variety of ways. For example, after the ethyl acetate and water from the top stream enter a phase separator, a water phase and an ester phase may be separated from each other, such as by fractional distillation, solvent extraction including acid-base extraction, or other liquid-liquid extraction method. The water phase may contain some amount of leftover ester and alcohol, and it can enter a recovery tower for further distillation. After the further distillation, the remaining water may be discharged from the bottom of the recovery tower as a waste stream.

The ester phase can be processed separately from the water phase. One part of the ester phase may flow back to the esterification reactor, and a remainder may enter a crude ester buffer tank to be preheated before entering a concentration tower. In the concentration tower, one or more final distillations may be carried out to produce a finished product with qualified ester content, acidity, and moisture.

During ethyl acetate production, corrosion metals may be generated at various points, including in the esterification reactor, due to the high temperatures involved and the presence of strongly acidic catalysts in the presence of water. Common materials used in ethyl acetate plants include stainless steels. Despite stainless steels' resistance to corrosion, the presence of strong acids like MSA can nevertheless attack the stainless steels and cause corrosion, leading to leaching of corrosion metals into the various process streams of the ethyl acetate production process. The high temperatures involved, along with the presence of water formed in the hydrolysis of acetic acid, increase the corrosion rate of the stainless steel equipment.

Conventionally, corrosion metals generated during ethyl acetate production are removed by removing a part of the bottom stream from the distillation column and diverting it to an evaporator tank instead of recycling it back to the esterification reactor, as shown in FIG. 1. In the evaporator tank, the purge stream is evaporated, and the vapor phase may be returned to the azeotropic distillation column, with optional additional cooling and/or condensation steps. The remaining heavy liquid along with corrosion metals is removed from the process as a heavy purge stream, as shown in FIG. 1.

In some embodiments, the corrosion metals may comprise a salt having a melting point in a range from 200° C. to 300° C. Non-limiting examples of the corrosion metal include Fe³⁺, Cr³⁺, and Ni²⁺. Other examples of corrosion metals may include ions or salts of aluminum, zinc, titanium, chromium, niobium, and/or molybdenum.

In some embodiments, the process streams, such as the bottom stream, can comprise corrosion metal impurities in an amount from 0.001 wt. % to 1.0 wt. %, based on the total mass of the process stream, such as from 0.001 wt. % to 0.5 wt. % or from 0.025 to 0.1 wt. %, or from 0.1 wt % to 0.2 wt %, or from 0.2 wt % to 0.3 wt %.

In addition to the corrosion metals, another challenge occurring in ethyl acetate production is that, during vaporization in the evaporator tank, the acid catalyst can decompose into sulfate ions. The sulfate ions can further convert into sulfuric acid vapors by reacting with water vapor, which is also present in the bottom stream. The combination of sulfuric acid, acetic acid and water, along with the high evaporation temperatures, such as 115° C. or higher, can corrode the evaporator tank and evaporator internals, such as the steam coils. Due in part to this sulfate corrosion, the evaporator typically has to be replaced very often.

The concentration of sulfur impurities and/or corrosion metals in the process streams, including the bottom stream, may vary, but generally is from 5 ppb to 500 ppm, e.g., 10 ppb to 100 ppb, or 100 ppb to 250 ppb, or 250 ppb to 500 ppb, or 500 ppb to 750 ppb, or 750 ppb to 1 ppm, or 1 ppm to 500 ppm, or 1 ppm to 200 ppm, by mass.

In the current invention disclosure, a modified process configuration is proposed to remove the corrosion metals using relatively low temperatures, such as 45° C. or less, such as from ambient atmospheric temperatures to 45° C.

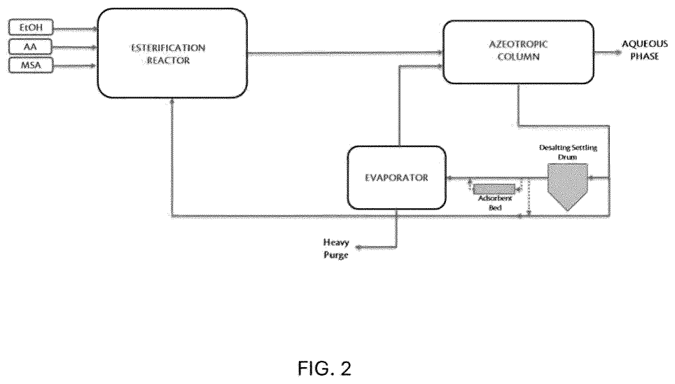

Some embodiments of the present disclosure relate to a method of producing ethyl acetate. The method of producing ethyl acetate of the present disclosure is shown diagrammatically as the process flow diagram illustrated in FIG. 2. Referring to FIG. 2, the method of producing ethyl acetate may include reacting ethanol, acetic acid, and an acid catalyst in an esterification reactor to form a product stream comprising ethyl acetate and at least one corrosion metal.

The esterification reactor is not particularly limited, and may be a batch reactor, a continuous stirred tank reactor (CSTR), or a plug flow reactor. In some embodiments, the esterification reactor may be a tower reactor, where the esterification occurs on multiple trays. In some embodiments, the esterification is a continuous reactor, with reactant streams being continuously added and product streams being continuously removed from the reactor. For example, tower reactors are a type of continuous reactor often used in esterification and other equilibrium-limited process. Generally, tower reactors are vertical columns, providing for efficient heat and mass transfer, including for continuous processes. They provide a large surface area for contact between the reactants. In some embodiments, the tower reactor may be equipped with packing materials to promote intimate contact between the liquid phase and vapor phase, which can enhance the reaction rate, while allowing improved separation of by-products, such as the water produced in the esterification reaction.

In some embodiment, the esterification reactor may be a direct addition reactor having one or more train reactors. For example, the direct addition reactor train may consists of one or more, such as two to four, or two to six, or two to eight, fixed-bed adiabatic reactors, which may be arranged in a radial flow configuration. The exotherm will depend upon the catalyst loading in each bed but is generally in the range from 5 to 15° C. across each catalyst bed. In some embodiments, the reaction takes place in the vapor phase and the inlet temperature to each train reactor is suitably about 90° C. to 130° C., such as 90° C. to 100° C., or 100° C. to 110° C., or 110° C. to 115° C., or 115° C. to 130° C., although this may be varied depending upon the condition of the catalyst and the flow rates of the acetic acid, ethanol, and acid catalyst.

An acetic acid stream may injected to the exit gases of the first reactors in the reactant trains to maintain the acetic acid to ethanol ratio at the entry point to each of the reactors within a predetermined range. Water may be added to these reactors to maintain control of the temperature of the gases fed to the next subsequent reactors in the system within a pre-determined range.

The esterification reactor may produce a product stream comprising ethyl acetate and at least one corrosion metal. The product stream may be distilled in an azeotropic distillation column. The distillation may form a top stream comprising the ethyl acetate and a bottom stream comprising at least one corrosion metal. The product stream may be continuously removed from the esterification reactor. The product stream may comprises ethyl acetate, water, and leftover reactants according to the equilibrium or steady-state conditions in the esterification reactor. The product stream may be removed from the top of the esterification reactor, the bottom of the esterification reactor, or from the side of the esterification reactor.

In some embodiments, the product stream enters a distillation column, such as an distillation column, as illustrated in FIG. 2. The distilling of the product stream may occur in the distillation column, and may form a top stream and a bottom stream. Generally, the top stream corresponds to a vapor phase or distillate of the distillation. For example, the top stream may be a liquid that is condensed from the vapor phase created by the distillation. Generally, the top stream is removed from an upper portion of the distillation column, such as the top of the distillation column. In some embodiments, the top stream is removed from the top half of the distillation column, or the top third, or the top quarter, or from a top surface of the distillation column. The top stream may be a liquid or it may be a vapor. If it is a vapor, then the vapor may be condensed in a separate condensation and/or cooling tank. In some embodiments, the top stream comprises ethyl acetate and water.

The distillation may be an extractive distillation. Extractive distillation is a method of separating azeotropes by carrying out the distillation in a multiplate distillation column in the presence of an added liquid, with the added liquid having a boiling point higher than the compounds being separated. The extractive agent is introduced near the top of the column and flows downward. Its presence on each plate of the distillation column alters the relative volatility of the close boiling compounds in a direction to make the separation on each plate greater, and thus requires either fewer plates to effect the same separation, or provides a greater degree of separation with the same number of plates. When the compounds to be separated form an azeotrope, the extractive agents will cause them to boil separately during the extractive distillation and thus make possible a separation that would not occur without extractive agent being present. The extractive agent should boil higher than any of the close boiling liquids being separated. In some embodiments, the extractive agent boils at least 20° C. higher than the lowest boiling component. Usually the extractive agent is introduced a few plates from the top of the column in insure that none of the extractive agent is carried over with the lowest boiling component.

At the bottom of a continuous distillation column, the less volatile components of the close boiling mixtures and the extractive agent are continuously removed from the column. The usual methods of separation of these two components are the use of another distillation column, cooling and phase separation, or solvent extraction.

In the esterification reactor, the product mixture may form a ternary azeotrope comprising ethyl acetate, ethanol, and water. The boiling point of the ternary azeotrope may be between 70° C. to 90° C., such as 75° C. to 80° C., 80° C. to 85° C., or 85° C. to 90° C. In some embodiments, the ternary azeotrope comprises at least 70% product ethyl acetate, at least 75% ethyl acetate, at least 80% ethyl acetate, or at least 90% ethyl acetate. In some embodiments, the extractive agent is water, in the form of steam. The steam may extract the ethanol from the ternary azeotrope. However, water has about 4% solubility in ethyl acetate and thus forms a minor azeotrope with it. Thus, both the ethanol and the ethyl acetate generally need to be removed as azeotropes by further distillation. Additional extractive agents can be used that both break the ethyl acetate-ethanol-water ternary azeotrope while also being easy to recover from the ethanol and water removed from the ethyl acetate. Non-limiting examples of suitable extractive agents include dimethylsulfoxide (DMSO) and N,N-dimethylformamide (DMFA), and other extractive agents described in U.S. Pat. No. 4,379,028, which is incorporated by reference herein in its entirety.

In some embodiments, the bottom stream is a distillation residue comprising the liquid portion of the product stream that was not vaporized during the azeotropic distillation. The bottom stream may contain unreacted reactants, such as ethanol, acetic acid, and the acid catalyst, and may comprise some portion of reaction products, including ethyl acetate, water, and by-products of the esterification reactant. The bottom stream may include at least one corrosion metal, where the corrosion metal has generally been formed by corrosion of the equipment, such as tanks, condensers, steam coils, and other equipment used in the ethyl acetate production process. The corrosion metals may be formed in the esterification reactor, the azeotropic distillation column, the evaporator, or any other component of the ethyl acetate production process where metal components are used. The corrosion metals can then travel throughout all of the process streams due to the recycling and recirculation of the processing streams. The bottom stream may be removed from the azeotropic column at the bottom half of the column, such as in the bottom third, or the bottom quarter. In some embodiments, the bottom stream is removed from a bottom surface of the azeotropic column.

For avoidance of doubt, the top stream is not required to be removed from a top portion of the azeotropic column, and the bottom stream is not required to be removed from a bottom portion of the azeotropic column, as the terms “top stream” and “bottom stream” are merely conventional terms corresponding to the distillate and the residue, respectively, and do not correspond to any spatial configuration of any tank or other equipment, unless otherwise specified. Thus, the top and bottom streams may be removed from any spatial location on the azeotropic column, without limitation.

In some embodiments, the method includes cooling the bottom stream in a settling tank to precipitate at least a first portion of the at least one corrosion metal from the bottom stream and form a supernatant stream. As illustrated schematically in FIG. 2, the settling tank may be a desalting settling drum. In some embodiments, the settling tank is a decanter tank, or a sedimentation basin, or a clarifier. The settling tank may include a heat exchanger to cool the bottom stream. In some embodiments, the bottom stream is cooled using a heat exchanger before entering the settling tank. Generally, settling tanks use gravity to separate heavy fluids from lighter fluids and to facilitate precipitation and separation of solids from a fluid phase.

In some embodiments, at least a part of the bottom stream is continuously routed from the distillation column into a settling tank having a volume of from 50 gallons to 200 gallons, such as 100 to 200 gallons, after cooling it to from 45° C. to atmospheric temperature, such as by a pump or gravity feed. The flow into the settling may be adjusted to achieve a holding time of the bottom stream in the settling tank of 1 to 10 hours, such as 2 to 8 hours, or 3 to 6 hours, or 4 to 5 hours.

In some embodiments, the settling tank has diagonal plates that redirect heavy components of the bottom stream toward the bottom of the settling tank, allowing faster precipitation of the corrosion metals, and allowing the supernatant stream to remain near the top of the settling tank. In some embodiments, both the inlet and the outlet of the settling tank are provided at or near the top of the tank, where the supernatant stream, which generally has lower density, is concentrated. In some embodiments, the supernatant stream passes out of a top portion of the settling tank and is moved through a sand filter to remove any remaining suspended precipitate particles. The precipitate including the corrosion metals may be drawn out of the bottom of the settling tank and pumped by a sludge pump, where it can then be disposed of in a variety of appropriate manners.

The settling tank is preferably a vessel which permits a holding time of the bottom stream ranging from 0.5-2 hours. The holding time may vary depending on the rate at which the bottom stream cools when it enters and resides in the settling tank. Preferably, the holding time is 0.5-1 hours at a temperature of 45° C. or less. The settling tank preferably has an inlet disposed at the top portion of the settling tank for addition of the bottom stream. As the bottom stream cools in the settling tank, it may pass over a system of weirs or interleaved plates that function to delay passage of the bottom stream from the inlet to the outlet of the settling tank, and maximize the time any particular portion of the bottom stream is present in the settling tank at a temperature of 45° C. or less. An outlet is preferably disposed at the bottom of the settling tank, which may have a conical shape. The outlet is preferably disposed at an elevation above an apex of the conical shape of the bottom of the settling tank. A second discharge pipe may be present at the apex of the conical settling tank to permit quick discharge of the settled materials.

In the settling tank, the bottom stream may form a precipitate including at least a portion of the corrosion metals. In the settling tank, the precipitation also forms a supernatant stream. In some embodiments, the supernatant stream does not include, or includes less of, the corrosion metals, relative to the bottom stream prior to entering the settling tank.

In some embodiments, the bottom stream is cooled to a temperature of 45° C. or less. The bottom stream may be cooled to a temperature of 40° C. or less, or 35° C. or less, or 30° C. or less, or 25° C. or less, or 20° C. or less.

In some embodiments, an entire portion of the bottom stream from the azeotropic distillation column enters the settling tank. In some embodiments, only a portion of the bottom streams enters the settling tank, such as 5% or less, 10% or less, 20% or less, 30% or less, 40% or less, 50% or less, 60 or less %, 70% or less, 80% or less, or 90% or less. The remainder of the bottom stream, which does not enter the settling tank, may be recycled back to the esterification reactor, or to an evaporator for purging, or divided between the esterification reactor and the evaporator, or sent to additional processing streams.

In some embodiments, the supernatant stream from the settling tank enters an adsorbent bed, as illustrated in FIG. 2. In the adsorbent bed, the supernatant stream may be contacted with a cation exchange resin to remove at least a portion of the remaining corrosion metals from the supernatant stream to form a reduced metal stream.

The adsorbent bed of the present method is not particularly limited, and may be a fixed-bed adsorber, or a moving or pulsed bed adsorber. Fixed-bed adsorbers generally include two or more towers filled with an adsorbent. While one bed is undergoing adsorption of the supernatant stream, the other bed is being regenerated to remove the adsorbed phase, thus removing the corrosion metals from the production process. In a moving bed or pulsed bed adsorber, the supernatant stream may enter the adsorbent bed from the bottom and flow up to the top of the bed, while fresh adsorbent may enter from the top and be removed from the bottom. In moving and pulsed beds, fresh adsorbent is continuously introduced and spent adsorbent is continuously removed from the bed.

The adsorbent used in the adsorbent bed of the present disclosure may include a cation exchange resin. The cation exchange resin is not particularly limited, and may include strong acid cationic resins or weak acid cationic resins. For example, strong acid cationic resins may include a polystyrene polymer that is conjugated to sulfonic acid functional groups. For example, weak acid cationic resins may include an acrylic polymer that is conjugated to carboxylic acid functional groups.

The cationic exchange resin may comprise a styrene-divinylbenzene polymer that is conjugated to strongly acidic functional groups. The strongly acidic functional groups may include sulfonic acid groups. In some embodiments, the cationic exchange resin may comprise an Amberlyst resin. Amberlyst resins are industrial-grade polymeric resins that are typically supplied in a bead form. In some embodiments, the cationic exchange resin may be an Amberlyst 15 resin. The resin beds may include any type of ion exchange resin known in the art. The resins used in the present invention may include metal exchanged functional groups as described in U.S. Pat. Nos. 5,220,558 and 4,615,806, the entireties of which are hereby incorporated by reference. In some embodiments, the ion exchange resin is functionalized with a co-precipitate to the impurity contained in the stream. The metal loading on the resin may vary, and preferably at least 1% of the active sites are occupied, and more preferably from 10 to 90% of the active sites, e.g., 30 to 70%. See, for example, U.S. Pat. No. 6,225,498, the entirety of which is incorporated herein by reference.

The resins useful for removing corrosion metal impurities from the process streams according to the present invention may include cation exchange resins either of the strong-acid or the weak-acid type. Both strong- and weak-acid type resins are readily available as commercial products. The weak-acid cation exchange resins are mostly copolymers of acrylic or methacrylic acids or esters or the corresponding nitriles, but a few of those marketed are phenolic resins. Preferably, strong-acid cation exchange resins are utilized. Strong-acid cation exchange resins predominantly comprise sulfonated styrene-divinylbenzene copolymers although some of the available resins of this type are phenol-formaldehyde condensation polymers. Amberlyst™ 15, Amberlite™ GT73, and Duolite ES 465 (Rohm and Haas) are exemplary commercial resins. The degree of cross linking in the styrene-divinylbenzene copolymers may vary and generally is from about 4% to 12%, preferably about 6 to 10%.

Either a gel type resin or a macroreticular type resin may be suitable, but the latter is preferred since organic components are present in the fresh acetic acid feed streams and recycled streams comprising acetic acid. Macroreticular resins are commonly employed in the catalytic art and require minimal water to maintain their swelling properties.

Other suitable guard bed materials are known in the art and include, for example, carbon, silica, alumina, ceramics, or combinations thereof. Ion exchange substrates comprising zeolites may also be employed, provided that the material is stable at the conditions of interest; that is, so long as the material will not chemically decompose or release silver or mercury in unacceptable amounts.

The corrosion metal impurity removal may be performed at temperatures ranging from 0° C. to 120° C., e.g., from 20° C. to 90° C., or from 10° C. to 45° C., or from 25° C. to 40° C.

Lower or higher temperatures are limited only by the stability of the resin to be employed. For some types of corrosion metals, such as chromium, removal may be more efficient at the higher temperatures. At the higher temperatures, a nitrogen or CO purge may be desirable.

The processes of the present invention may be carried out in any suitable resin bed configuration. The flow rates of acetic acid feed streams or recycle stream may vary, but generally the flow through the resin bed may be from about 0.5 to about 50 bed volumes per hour (BV/hr), e.g., from about 1 to about 30 BV/hr, wherein a bed volume as used herein is simply the volume of the resin bed.

In some embodiments, all of the supernatant stream enters the adsorbent bed. In some embodiments, only a portion of the supernatant stream enters the adsorbent bed, and a remainder may be recycled to the esterification reactor and/or to the evaporator for corrosion metals purging, or sent to additional processing streams. In some embodiments, 5% or less, 10% or less, 20% or less, 30% or less, 40% or less, 50% or less, 60 or less %, 70% or less, 80% or less, or 90% or less of the supernatant stream enters the adsorbent bed having the cation exchange resin.

In some embodiments, as illustrated in FIG. 2, the reduced metal stream enters an evaporator tank for corrosion metals purging. The evaporator tank generally operates in a continuous mode to evaporate a portion of the supernatant stream and precipitate out corrosion metals, where they are removed from the production process as a waste stream. After purging in the evaporator tank, at least a portion of the reduced metal stream may be returned to the azeotropic column, as shown in FIG. 2. In some embodiments, at least a portion of the reduced metal stream is recycled to the esterification reactor.

In some embodiments, the cooling of the bottom stream and/or the contacting of the supernatant stream with the cation exchange resin removes at least 50% of the corrosion metals. In some embodiments, it removes at least 60%, or at least 70%, or at least 80%, or at least 90%, or at least 95%, or at least 99%, of the corrosion metals by mass, relative to an initial concentration of the corrosion metals in the bottom stream prior to the cooling and the contacting with the supernatant stream.

In some embodiments, the bottom stream comprises iron sulfate ions, and the cooling of the bottom stream precipitates at least 50% of the iron sulfate ions, such as at least 60%, or at least 70%, or at least 80%, or at least 90%, or at least 95%, of the iron sulfate ions.

In some embodiments, the acid catalyst comprises a sulfonic acid. In some embodiments, the sulfonic acid is an alkane sulfonic acid such as methanesulfonic acid (MSA). In some embodiments, the acid comprises sulfuric acid or nitric acid.

In some embodiments, the flow rate of one or more streams in the ethyl acetate production process may be adjusted based on an initial concentration of corrosion metals in one or more streams, such as the initial concentration present in the bottom stream at the point of exit from the distillation column, prior to any cooling, wherein the flow being adjusted may be the same or different as the flow having the concentration of corrosion metals. In some embodiments, a flow rate of the bottom stream from the distillation column to the settling tank may be adjusted based on the concentration of corrosion metals in the bottom stream. In some embodiments, the adjusting includes decreasing the flow rate if the concentration of the corrosion metals increases, and increasing the flow rate if the concentration of the corrosion metals decreases, relative to the initial concentration of the corrosion metals. In some embodiments, the adjusting includes increasing the flow rate if the concentration of the corrosion metals increases, and decreasing the flow rate if the concentration of the corrosion metals decreases.

Settling tanks may be either rectangular or circular in design. Settling tanks with rectangular basins are hydraulically more stable, and flow control for large volumes is easier with this configuration. In some embodiments, the ratio of length to width in the settling tank may be from 2:1 to 5:1, such as 2:1, 3:1, 4:1, or 5:1. In some embodiments, the bottom can be sloped to facilitate sludge removal. A slow-moving mechanical sludge scraper may be used to continuously pull the settled material, including the corrosion metals, into a sludge hopper for periodic pumping out of the production process.

In some embodiments, the settling tank is a circular basin settling tank. The input flow enters at the center of the settling tank and is baffled to flow radially towards the perimeter, with the horizontal velocity of the liquid continuously decreasing as the distance from the center increases. Sludge remove mechanisms in circular tanks are simpler and require less maintenance than in long rectangular tanks.

During the precipitation process, the bottom stream may be continuously fed into the settling tank at the top, where the suspended particles are allowed to settle under the influence of gravity to form a thickened particle bed on the bottom of the tank. This precipitate is normally removed by a pump at the base of the settling tank, and either processed or disposed of. The supernatant stream, with minimal suspended particles, is removed from the top of the tank.

The settling tank may have an overflow rate, which is a rate at which the bottom stream enters the settling tank. The overflow rate according to some embodiments may be 10 gallons per minute per square foot (g/m*ft²) or less, such as 5 g/m*ft² or less, 2.5 g/m*ft² or less, 1.0 g/m*ft² or less, 0.5 g/m*ft² or less, or 0.1 g/m*ft² or less.

In some embodiments of the method described herein, a portion of the bottom stream going to the evaporator tank is cooled using a heat exchanger to form a cooled bottom stream, and enters settling tank and/or an adsorbent bed comprising a cation exchange resin. After cooling, this stream is sent to an intermediate storage vessel, such as a settling tank, where at least a portion of the corrosion metals are precipitated. In some embodiments, a supernatant stream from the settling tank enters an adsorption tower comprising a cation exchange resin, such as a strongly acidic, macroreticular polystyrene-based ion exchange resin, for example Amberlyst 15. In some embodiments, the resin can be a Purolite resin, such as Puroline-CT252 or Purolite-CT276. In some embodiments, the cation exchange resin can be a functionalized zeolite, where the zeolite can be H-Beta, USY-H, and H-mordenite.

In some embodiments, all of the remaining corrosion metal ions, or at least a portion of the remaining corrosion metal ions, such as Fe³⁺, Cr³⁺ and Ni²⁺, are adsorbed on the cation exchange resin, forming a clear liquid that leaves the column, where the clear liquid may be a reduced metal stream. Based on experimental studies, more than 95% of corrosion metals can be removed by the adsorption column, leaving a very clean reduced metal stream having very low amounts of metal ions.

In addition to removing corrosion metals, some embodiments of the present method also remove free sulfate ions from one or more streams in the production process. For example, by cooling the bottom stream from the distillation column to ambient temperature, such as to an ambient temperature in a range of from 30° C. to 40° C., or from 35° C. to 40° C., more than 70% of the iron can be removed in the form of ferrous sulfate (FeSO₄·xH₂O). As sulfate ions are known for their high corrosion potential to steel equipment, this reduction of sulfate ions can significantly reduce the corrosion rate in ethyl acetate production.

Some embodiments provide for cooling one or more reaction mixtures of ethyl acetate production, such as the bottom stream, to a temperature of about 45° C. or less. This cooling can remove corrosion metal salts that have high melting points, such as in the range of 200° C. to 300° C. In some embodiments, the corrosion metal salts have melting points in the range of 100° C. to 200° C., or from 50° C. to 100° C., or from 20° C. to 50° C. In some embodiments, a cation exchange resin is used to remove multivalent corrosion metal ions, such as Fe³⁺, Cr³⁺, and/or Ni²⁺, in an adsorption column.

In some embodiments, a flow rate to the corrosion metal removal section of the plant can be adjusted depending at least in part on the corrosion metal concentration in one or more process streams, such as in the bottom stream of the azeotropic distillation column.

In some embodiments of the method described herein, technical benefits can include a reduced corrosion rate in the evaporator section of the plant, due to reduced sulfate ion accumulation, and/or reduced multivalent corrosion metal concentrations. For example, the reduced corrosion rate of a stainless steel tank, such as the evaporator tank, may be at most 10 mm/year at 100° C., at most 5 mm/year, at most 1 mm/year, at most 0.5 mm/year, at most at most 0.1 mm/year, or at most 0.01 mm/year. In some embodiments, the corrosion rate of the stainless steel tank may be reduced by 70% relative to a similar process which does not cool the bottom stream to precipitate corrosion metals and/or expose the supernatant stream to a cationic exchange resin, such as reduced by 60%, 50%, 40%, 30%, 20%, 10%, or 5%. Typical acceptable corrosion rates for both the azeotropic distillation, the evaporator tank and the recycle stream pipelines are less than from about 0.025 mm/year to about 0.2 mm/year, such as less than about 0.025 mm/year, less than about 0.05 mm/year, less than about 0.075 mm/year, less than about 0.1 mm/year, less than about 1.5 mm/year, or less than about 0.2 mm/year. The corrosion rates can be measured through weight-loss over time, such as by using carefully placed, retrievable test coupon inserts.

This can increase the usable lifetime of equipment in the production process, reducing the cost of production and reducing the frequency of required maintenance and replacement.

In some embodiments, the purge steam flow can be reduced significantly, as all or most of the corrosion metals are removed using low-temperature processes, as described herein, rather than high-temperature evaporation as used traditionally. The reduction of the purge stream flow can be achieved at last in part by reducing a flow amount entering the evaporator tank for metals corrosion purging. As this part of the production process can have the highest contribution to formation of corrosion metals, the present method can also reduce the initial formation of corrosion metals in the production process. Thus, as described herein, the ethyl acetate production process can also be more flexible in terms of adjusting the flow to the purging section depending on corrosion metal analysis results. In some embodiments, the flow to the purging section can be reduced by 70%, by 60%, by 50%, by 40%, by 30%, by 20%, by 10%, or by 5% relative to a process which does not cool the bottom stream to precipitate carrion metals and/or expose the supernatant stream to a cationic exchange resin.

Numerous modifications and variations of the present disclosure are possible in light of the above teachings. It is therefore to be understood that within the scope of the appended claims, the invention may be practiced otherwise than as specifically described herein.

1. A method of producing ethyl acetate, the method comprising:
reacting ethanol, acetic acid, and an acid catalyst in an esterification reactor to form a product stream comprising the ethyl acetate and water;
distilling the product stream in a distillation column to form a top stream comprising the ethyl acetate, ethanol, and a water azeotrope, and a bottom stream comprising acetic acid, ethyl acetate, ethanol, water, the acid catalyst, and at least one corrosion metal having an initial corrosion metal concentration in the bottom stream;
cooling at least a portion of the bottom stream in a settling tank to precipitate at least a first portion of the at least one corrosion metal from the bottom stream and form a supernatant stream;
contacting at least a portion of the supernatant stream with a cation exchange resin to remove at least a second portion of the at least one corrosion metal from the supernatant stream and form a reduced metal stream;
evaporating at least a portion of the reduced metal stream in an evaporator tank to purge at least a third portion of the at least one corrosion metal from the reduced metal stream and form a purged stream; and
returning at least a portion of the purged stream to the distillation column,
wherein:
the bottom stream is cooled to a temperature of about 45° C. or less in the settling tank, and
the cooling of the bottom stream and the contacting of the supernatant stream with the cation exchange resin removes at least 75% of the at least one corrosion metal relative to the initial corrosion metal concentration in the bottom stream.

2. The method of claim 1, further comprising:
adjusting a flow rate of the bottom stream into the settling tank based on the initial corrosion metal concentration in the bottom stream.

3. The method of claim 2, wherein the adjusting of the flow rate comprises decreasing the flow rate if a concentration of the at least one corrosion metal increases relative to the initial corrosion metal concentration in the bottom stream.

4. The method of claim 1, wherein the bottom stream further comprises iron sulfate ions having an initial iron sulfate concentration, and the cooling of the bottom stream precipitates at least 50% of the iron sulfate ions relative to the initial iron sulfate concentration.

5. The method of claim 4, wherein cooling of the bottom stream precipitates at least 70% of the iron sulfate ions relative to the initial iron sulfate concentration.

6. The method of claim 1, wherein the acid catalyst comprises a sulfonic acid.

7. The method of claim 6, wherein the sulfonic acid is methanesulfonic acid (MSA).

8. The method of claim 1, wherein the cation exchange resin comprises a sulfonated styrene-divinylbenzene copolymer.

9. The method of claim 8, wherein a degree of cross linking in the sulfonated styrene-divinylbenzene copolymer is from 4% to 12%.

10. The method of claim 1, wherein the distillation column is an azeotropic distillation column.

11. The method of claim 1, wherein the top stream of the distillation column further comprises water.

12. The method of claim 10, wherein the top stream of the distillation column further comprises ethanol.

13. The method of claim 1, wherein the at least one corrosion metal comprises a metal salt having a melting point of from 200° C. to 300° C.

14. The method of claim 1, wherein the at least one corrosion metal comprises one or more of Fe³⁺, Cr³⁺, and Ni²⁺.

15. The method of claim 1, wherein the bottom stream of the distillation column is cooled to a temperature of 40° C. or less.

16. The method of claim 1, wherein the cooling of the bottom stream and the contacting of the bottom stream with the cation exchange resin removes at least 90% of the at least one corrosion metal relative to the initial corrosion metal concentration in the bottom stream.

17. The method of claim 1, wherein the cooling of the bottom stream and the contacting of the bottom stream with the cation exchange resin removes at least 95% of the at least one corrosion metal relative to the initial corrosion metal concentration in the bottom stream.

18. The method of claim 1, wherein the reacting of the ethanol, acetic acid, and the acid catalyst is performed at a temperature of 90° C. or higher.

19. The method of claim 11, wherein at least a portion of the purged stream from the evaporator tank is returned to the distillation column.

20. The method of claim 1, wherein at least a portion of the supernatant stream is returned to the esterification reactor.
